(19) United States
(12) Patent Application Publication
(10) Pub. No.: US 2007/0098670 A1
(43) Pub. Date: May 3, 2007

(54) Compositions and methods for using juice organic, juice based skin care products

(76) Inventors: Melissa Jochim, Orinda, CA (US); Christin Powell, Sausalito, CA (US); Karen Behnke, San Anselmo, CA (US)

Correspondence Address: BAKER & MCKENZIE LLP;PATENT DEPARTMENT, 2001 ROSS AVENUE, SUITE 2300, DALLAS, TX, 75201, US

(21) Appl. No.: 11/264,549

(22) Filed: Nov. 1, 2005

(51) Int. Cl. A61K 8/97; A61K 36/752; A61K 36/886; A61K 36/899; A61K 36/87; A61K 36/73

(52) U.S. Cl. 424/74; 424/195.17; 424/736; 424/765; 424/771; 424/766; 424/750; 424/195.18; 424/744

(57) ABSTRACT

The disclosure herein is directed to organic skin-based products that comprise an excess of 70% organic ingredients. The skin care products described herein are composed of a plurality of juices and other organic materials.

### BACKGROUND

1. Field of the Invention

This disclosure relates to skin care products, and more particularly to juice based skin care compositions and methods for using juice based skin care products.

2. Background of the Invention

There are hundreds of skin care products on the market today designed to provide a variety of benefits and care for hands, feet, body, eyes, face, etc. The effectiveness of these skin care products varies widely, however, and in addition to being ineffective, many of them can have adverse side effects and can even damage the skin. This is especially true for sensitive and/or blemish-prone skin.

The main ingredient in all conventional skin care products is water. In fact, over 50% of many conventional skin care products can be made up of water. Further, conventional skin care product can also include artificial fillers, preservatives, tars, petroleum, synthetic fragrances, and parabens. All of these ingredients are absorbed by the skin when the skin care product is applied, and can cause such problems as blemishes, dry skin, rashes, etc.

### SUMMARY

The disclosure herein is directed to organic skin-based products that comprise an excess of 70% organic ingredients. For example, the skin care products described herein are composed of a plurality of juices and other organic materials.

In one aspect, the organic skin care products described can be used in a variety of skin care solutions to achieve various skin care objectives.

These and other features, aspects, and embodiments of the invention are described below in the section entitled “Detailed Description.”

### BRIEF DESCRIPTION OF THE DRAWINGS

Features, aspects, and embodiments of the inventions are described in conjunction with the attached drawings, in which:

FIG. 1 is a flowchart illustrating one example skin care solution that makes use of organic skin care products in accordance with one embodiment;

FIG. 2 is a flowchart illustrating another example skin care solution that makes use of organic skin care products in accordance with another embodiment; and

FIG. 3 is a flowchart illustrating another example skin care solution that makes use of organic skin care organic products in accordance with still another embodiment.

### DETAILED DESCRIPTION OF THE PREFERRED EMBODIMENTS

The embodiments described below are directed to compositions for organic skin care products. The percentage of organic ingredients in each of the compositions is an excess of 70% in accordance with the USDA National Organic Program food standards. In particular, the main ingredient in each of the compositions is organic fruit juice. The organic fruit juice is used in place of water as used in conventional skin care products. By using organic fruit juices in the place of water, the compositions below can achieve the 70% or greater USDA standard. In fact, the FDA will not recognize any water-based skin care product as an organic product.

Using organic juices as a base composition for a skin care product is a costly process with many challenging logistics. For example, seasonal produce, weather sensitivity and the fact that each crop has unique chemistry and colors combine to make the development of juice-based organic skin care products extremely difficult; however, the compositions described below have been shown to produce repeatable results.

Each of the compositions below has a unique blend of organic juices with a specific benefit for the skin type being targeted; however, the same juices, such as apple, white grape, red grape, lemon juice, to name just a few, are used in each of the compositions. In fact, up to 17 organic juices can be found in the compositions described herein. Organic juices are rich in protein antioxidants, essential vitamins, vital phyto-nutrients, and powerful hydroxy acids, making them excellent ingredients for skin care products. The compositions below result in skin care products that are quite concentrated, but beneficial for the skin. It will be clear that the compositions below do not make use of any artificial fillers, toxic preservatives, water to dilute the compositions, tars, petroleum, synthetic fragrances, or parabens.

The following descriptions are related to several example skin care products and compositions. The following examples should not be seen as limiting the products and compositions described herein to any particular products and compositions however. It will be understood, that many more products and compositions are possible. These new products and compositions can comprise minute to substantial variations on the products and compositions described herein, or can comprise entirely new products and compositions. Thus, the products and compositions described below are presented by way of example only.

A first example of an organic, juice-based skin care product is an antioxidant moisturizer. In one embodiment, an organic, juice-based antioxidant moisturizer can include one or more of the following organic ingredients: an organic juice solution, e.g., a pyrus malus (apple) juice, citrus medica limonum (lemon) juice, vitis vinifera (white grape) juice, and aloe barbadensis (leaf) juice. Further, the ingredients can include organic rose canina (rosehip) fruit extract; organic camellia sinensis (green tea) leaf extract; glycerin; organic plant oils, e.g., of butyrospermum parkii (shea butter) and simmondsia chinensis (jojoba) seed; organic essential fatty acids, e.g., of oenothera biennis (evening primrose), linum usitatissimum (linseed) seed, and borago officinali (borage) seed; organic honey; vitus vinifera (grape) seed oil; organic algae extract; squalane; limnanthes alba (meadowfoam) seed oil; tocopheryl acetate (vitamin E); retinyl palmitate (vitamin A); ubiquinone (coenzyme Q10); thioctic acid (alpha-lipoic acid); dipeptide-2 and palmitoyl tetrapeptide-3; magnesium ascorbyl phosphate (vitamin C); pathenol (vitamin B5); dimethylaminoethanol (DMAE); hyaluronic acids; hydroxypropyl starch phosphate; glyceryl stearate; potassium sorbate; phospholipids; beta carotene; benzyl alcohol; palmitic acid; stearic acid; cetearyl glucoside; xanthan gum; methylisothiazolinone; disodium edta; sodium hydroxide; and phenoxyethanol.

Moreover, the ingredients described above can include blends of pure essential oils produced by and for Juice Beauty, Inc. located in California, 94901 and at the website www.juicebeauty.com. These pure essential oils can include May Cheng, Ho Wood and Mandarin pure essential oils. These pure essential oils can be included in a ratio of 2 to 1 to 3.

The ingredients listed above can be used to compose an SPF 30 or 15 moisturizer with lower levels of AHA. Importantly, no parabens are used in the composition of a moisturizer using the ingredients listed above. Further, the ingredients do not contain any harsh cleansers, which can have adverse effects on the skin. The ingredients are combined in the manner to produce a moisturizer that is at least 70% organic and is high as even 80% organic.

Another example of an organic, juice-based product can be an AHA antioxidant serum. In one embodiments, such an AHA antioxidant serum can comprise an organic alpha, betahydroxy acid juice solution, e.g., comprising pyrus malus (apple) juice (malic), citrus medica limonum (lemon) juice (citric), vitus vinifera (white grape) juice (tartaric), organic raw cane sugar (glycolic), organic willow bark extract (salicylic), organic milk peptides (lactic), organic aloe barbadensis (leafjuice).

Further, the ingredients can include glycerin; organic camellia sinensis (green tea) leaf extract; organic algae extract; glycerin, vitus vinifera (organic grape) seed oil; organic essential fatty acids, e.g., oenothera biennis (evening primrose), linum usitatissimum (linseed) seed, and borago officinali (borage) seed; ubiquinone (coenzyme Q10); thioctic acid (alpha-lipoic acid); dipeptide-2 and palmitoyl tetrapeptide-3; magnesium ascorbyl phosphate (vitamin C); tocopheryl acetate and tocopherol (vitamin E); retinol palmitate (vitamin A); sclerotium gum; dimethylaminoethanol (DMAE); phenoxyethanol; sodium hydroxide; benzyl alcohol; disodium edta; phospholipids; hyaluronic acids; and potassium sorbate.

Further, the antioxidant serum described above can include juice beauty essential oils, such as May Cheng, Ho Wood and Mandarin. The pure essential oils can be combined in a ratio of 1 to 1 to 5.

Further, as noted azulene can be added to the ingredients in order to give the serum a green apple appearance.

Another example antioxidant serum can comprise an organic juice solution of vitis vinifera (white grape) juice, citrus aurantium dulcis (orange) juice, and aloe barbadensis leaf juice. The serum can further comprise glycerin; vitis vinifera (organic grape) seed oil; organic essential fatty acids of oenothera biennis (evening primrose); linum usitatissimum (linseed) seed and borago officinali (borage) seed; organic algae extract; ubiquinone (coenzyme Q10); thioctic acid (alpha-lipoic acid); magnesium ascorbyl phosphate (vitamin C); tocopheryl acetate and tocopherol (vitamin E); retinyl palmitate (vitamin A); sclerotium gum; phenoxyethanol; sodium hydroxide; benzyl alcohol; disodium edta; phospholipids; hyaluronic acid; dimethylaminoethanol (DMAE); potassium sorbate; dipeptide-2; and palmitoyl tetrapeptide-3.

Further, the antioxidant serum described above can include juice beauty essential oils, such as Amyris and May Chang. The pure essential oils can be combined in a ratio of 2 to 1.

Eye cream is another example of an organic, juice-based skin care product. In one embodiment, such an eye clean can comprise an organic juice solution, e.g., of pyrus malus (apple) juice, daucus carota sativa (teric) juice, and aloe barbadensis leaf juice; organic cucumis sativus (cucumber) extract; organic plant oils, e.g., of helianthus annuus (sunflower) and persea gratissima (avocado); camellia sinensis (organic green tea) leaf extract; organic essential fatty acids of e.g., oenothera biennis (evening primrose), linum usitatissimum (linseed) seed, and borago officinali (borage) seed; glycerin; limnanthes alba (meadowfoam) seed oil; phytonadione (vitamin K1)); retinol palmitate (vitamin A); magnesium ascorbyl phosphate (vitamin C); tocopheryl (vitamin E); cetearyl glucoside; ubiquinone (coenzyme Q10); thioctic acid (alpha-lipoic acid); DMAE; dipeptide-2; palmitoyl tetrapeptide-3; phenoxyethanol; hesperidin methyl chalcone (vitamin P); sodium hyaluronate; cetearyl alcohol; benzyl alcohol; potassium sorbate; steareth-20; xanthan gum; tetrasodium edta; sodium hydroxide; azulene; citrus aurantium (orange blossom); and pure essential oils.

The eye cream ingredients described above can be used to compose a powerful complex of age-defying DMAE, vitamin antioxidants, alpha lipoic acid and co-enzyme Q10, combined with essential nutrients and peptides to hydrate, smooth lines, and help firm the area around the eyes.

Further, as noted azulene and apple juice can be added to the ingredients in order to give the eye cream a green apple smell and appearance.

Cleansing milk is another example of an organic, juice-based skin care product. In one embodiment, such cleansing milk can comprise an organic juice solution of vitis vinifera (white grape) juice, rubus idaeus (red raspberry) juice, and aloe barbadensis leaf juice. The ingredients can further comprise organic botanical extracts, e.g., of calendula officinalis flower, matricaria chamomilla flower, and tilia europea (linden) leaf; glycerin; organic plant oils, e.g., of carthamus tinctorius (safflower) seed, prunus amygdalus dulcis (sweet almond), helianthus annuus (hybrid sunflower), sesamum indicum (sesame), and vitis vinifera (grape) seed oil; caprylic/capric triglyceride; cetearyl alcohol; cetearyl glucoside; tocopheryl acetateand tocopherol (vitamin E); sorbitan stearate; retinyl palmitate (vitamin A); phospholipids; ascorbyl palmitate (vitamin C); tetrasodium edta; panthenol (vitamin B5); allantoin; phenoxyethanol; xanthan gum; benzyl alcohol; potassium sorbate; and citric acid.

Further, the cleansing milk product described above can include juice beauty essential oils, such as Sweet Orange, Chamomile, Frankincense, and Orange Blossom/Neroli. The pure essential oils can be combined in a ratio of 6 to 1 to 1 to 2.

Lip tints can be another example of an organic, juice-based skin care product. In one embodiment, an organic, juice based lip tint can comprise an active ingredient of titanium dioxide, as well as organic beeswax; organic sunflower oil; organic sea algae; organic lecithin; organic jojoba oil; organic coconut oil; organic aloe vera; tocopheryl (vitamin E); retinyl palmitate (vitamin A); ascorbyl palmitate (vitamin C); organic honey; organic propolis; and natural flavors, such as peach and guava.

The lip tint can also comprise natural tints that can include, e.g., mica and iron oxides, but no fdc or carmine.

One particular example lip tint can specifically comprise cocos nucifera (organic coconut) oil; helianthus annuus (organic sunflower) oil; butyrospermum parkii (organic shea butter) fruit; (organic) beeswax, ozokerite wax; euphorbia cerifera (organic candelilla) wax; copernicia cerifera (organic camauba) wax; simmondsia chinensis (organic jojoba) seed oil; panthenol (pro-vitamin B5); lecithin; (organic) honey; ascorbyl palmitate (vitamin C palmitate); aloe barbadensis (organic aloe vera) leafjuice; algae (seaweed) extract; psidium guajava (organic guava) fruit extract; prunus persica (organic peach) kernel oil; tocopherol (antioxidant); mica; iron oxides; titanium dioxide; and sucralose

Further, this embodiment of a lip tint can comprise as active ingredients: octinoxate (octyl methoxycinnamate) and oxybenzone (benzophenone-3).

Another particular example lip tint can specifically comprise cocos nucifera (organic coconut) oil, helianthus annuus (organic sunflower) oil, Butyrospermum Parkii (Organic Shea Butter) Fruit, (Organic) Beeswax, Ozokerite Wax, Euphorbia Cerifera (Organic Candelilla) Wax, Copernicia Cerifera (Organic Carnauba) Wax, Simmondsia Chinensis (Organic Jojoba) Seed Oil, Panthenol (Pro-Vitamin B5), Lecithin, (Organic) Honey, Ascorbyl Palmitate (Vitamin C Palmitate), Aloe Barbadensis (Organic Aloe Vera) Leaf Juice, Algae (Seaweed) Extract, Psidium Guajava (Organic Guava) Fruit Extract, Prunus Persica (Organic Peach) Kernel Oil, Tocopherol (Antioxidant), Mica, Iron Oxides, Titanium Dioxide, Sucralose

The lip tint described above can further comprise active ingredients of: Octinoxate (Octyl Methoxycinnamate), Oxybenzone (Benzophenone-3)

Another particular example lip tint can specifically comprise: Cocos Nucifera (Organic Coconut) Oil, Helianthus Annuus (Organic Sunflower) Oil, Butyrospermum Parkii (Organic Shea Butter) Fruit, (Organic) Beeswax, Ozokerite Wax, Euphorbia Cerifera (Organic Candelilla) Wax, Copernicia Cerifera (Organic Carnauba) Wax, Simmondsia Chinensis (Organic Jojoba) Seed Oil, Panthenol (Pro-Vitamin B5), Lecithin, (Organic) Honey, Ascorbyl Palmitate (Vitamin C Palmitate), Aloe Barbadensis (Organic Aloe Vera) Leaf Juice, Algae (Seaweed) Extract, Psidium Guajava (Organic Guava) Fruit Extract, Prunus Persica (Organic Peach) Kernel Oil, Tocopherol (Antioxidant), Mica, Iron Oxides, Titanium Dioxide, Sucralose

The lip tint described above can further comprise active ingredients of: Octinoxate (Octyl Methoxycinnamate) and Oxybenzone (Benzophenone-3).

Another particular example lip tint can specifically comprise: Helianthus Annuus (Organic Sunflower) Seed Oil, Cocos Nucifera (Organic Coconut) Oil, Copernicia Cerifera (Carnauba) Wax, Ricinus Communis (Castor) Seed Oil, Euphorbia Cerifera (Organic Candelilla) Wax, (Organic) Beeswax, Mica, Simmondsia Chinensis (Organic Jojoba) Seed Oil, Panthenol (Pro-Vitamin B5), Lecithin, (Honey) Honey, Butyrospermum Parkii (Organic Shea Butter) Extract, Bee Propolis Extract, Ascorbyl Palmitate (Vitamin C Palmitate), Aloe Barbadensis (Organic Aloe Vera) Leaf Juice, Algae (Seaweed) Extract, Psidium Guajava (Guava) Fruit Extract, Prunus Persica (Peach) Kernel Oil, Tocopherol (Antioxidant), Tocopheryl Acetate (Vitamin E Acetate), Sucralose, Propylparaben, Titanium Dioxide

The lip tint describe above can further comprise active ingredients of: Octinoxate (Octyl Methoxycinnamate) and Oxybenzone (Benzophenone-3).

Another particular example lip tint can specifically comprise: Helianthus Annuus (Organic Sunflower) Seed Oil, Cocos Nucifera (Organic Coconut) Oil, Copernicia Cerifera (Camauba) Wax, Ricinus Communis (Castor) Seed Oil, Euphorbia Cerifera (Organic Candelilla) Wax, (Organic) Beeswax, Mica, Simmondsia Chinensis (Organic Jojoba) Seed Oil, Panthenol (Pro-Vitamin B5), Lecithin, (Honey) Honey, Butyrospermum Parkii (Organic Shea Butter) Extract, Bee Propolis Extract, Ascorbyl Palmitate (Vitamin C Palmitate), Aloe Barbadensis (Organic Aloe Vera) Leaf Juice, Algae (Seaweed) Extract, Psidium Guajava (Guava) Fruit Extract, Prunus Persica (Peach) Kernel Oil, Tocopherol (Antioxidant), Tocopheryl Acetate (Vitamin E Acetate), Sucralose, Propylparaben, Titanium Dioxide

The lip tint describe above can further comprise active ingredients of: Octinoxate (Octyl Methoxycinnamate) and Oxybenzone (Benzophenone-3).

Lip plumber is another example of an organic, juice-based skin care product. In one embodiment, such a lip plumber product can comprise an organic juice solution of citrus aurantium dulcis (orange) juice and aloe barbadensis leaf juice, as well as organic simmondsia chinensis (jojoba) seed oil; glycerin; organic honey; organic essential fatty acids of oenothera biennis (evening primrose); linum usitatissimum (linseed) seed & borago officinali (borage) seed; tocopheryl acetate & tocopherol (vitamin E); retinyl palmitate (vitamin A); ascorbyl palmitate (vitamin C); caprylic/capric triglyceride; ethylhexyl (octyl) palmitate; tribehenin; sorbitan isostearate; palmitoyl oligopeptide; glyceryl stearate; sorbitan stearate; phospholipids; glycolic acid; palmitic acid, stearic acid; cetearyl glucoside; xanthan gum; and potassium sorbate.

Further, the lip plumber product described above can include juice beauty essential oils, such as citrus tangerina and cinnamomum cassia leaf (cinnamon) pure essential oils. Such a lip plumber can also comprise other Juice Beauty pure essential oils.

Another example organic, juice based product is a blemish clearing serum composed so as to reduce inflammation and prevent and heal blemishes. Such a blemish clearing product can comprise an organic juice solution of pyrus malus (apple) juice, citrus medica limonum (lemon) juice, and aloe barbadensis leaf juice, as well as organic raw cane surgar (glycolic acid); glycerin; organic botanical extracts of taraxacum officinale (dandelion) leaf,; Melissa officinalis (lemon balm) leaf and salvia officinalis (sage) leaf. Such a blemish clearing cream can also comprise organic camellia sinensis (green tea) leaf extract; organic algae extract; ubiquinone (coenzyme q10); magnesium ascorbyl phosphate (vitamin C); tocopheryl acetate (vitamin E); retinyl palmitate (vitamin A); panthenol (vitamin B5); allantoin; phospholipids; sodium hydroxide; sclerotium gum; phenoxyethanol; benzyl alcohol; potassium sorbate; tetrasodium edta; and xanthan gum. Such a lip plumber can also comprise Juice Beauty pure essential oils.

Another example of an organic, juice-based product is a hydrating nutrient mask that can be composed to deliver essential fatty acids to balance protective surfaces on exfoliated skin. Such a hydrating nutrient mask can comprise an organic juice solution of, e.g., pyrus malus (apple) juice, citrus medica limonum (lemon) juice, vitis vinifera (white grape) juice, and aloe barbadensis leaf juice; organic essential fatty acids of oenothera biennis (evening primrose), linum usitatissimum (linseed) seed, and borago officinali (borage) seed; organic honey; vitis vinifera (grape) seed oil; glycerin; sodium hyaluronate; organic honey; apple pectin; organic rose canina (rosehip) fruit extract; organic camellia cinensis (green tea) leaf extract; organic sea algae; caprylic/capric triglyceride; glycerol stearate; stearic acid; sodium PCA; ubiquinone (coenzyme Q10); thioctic (alpha-lipoic acid); dipeptide-2; palmitoyl tetrapeptide-3; magnesium ascorbyl phosphate (Vitamin C); tocopherol acetate (Vitamin E); and retinol palmitate (Vitamin A).

Further, the ingredient for the hydrating nutrient mask described above can include juice beauty pure essential oils, such as May Cheng, Ho Wood, and Mandarin. The pure essential oils can be included in ratio of the 2 to 1 to 3.

Further, as noted azulene can be added to the ingredients in order to give the serum a green apple appearance.

Another example of an organic, juice-based product is an exfoliating cleanser that can be composed to remove dull skin cells and brighten and refine skin texture. Such an exfoliating cleanser can comprise an organic juice solution of vitis vinifera (white grape) juice, ananas sativus, (pineapple) juice, and aloe barbadensis leaf juice, as well as organic plant oils of sesamum indicum (sesame) seed; prunus amygdalus; dulcis (sweet almond); butyrospermum parkii (shea butter); persea gratissima (avocado) and simmondsia chinensis (jojoba wax) seed; organic botanical extracts of calendula officinalis flower; matricaria chamomilla flower and tilia europea (linden) leaf; caprylic/capric triglyceride; glycerin; organic honey; organic algae extract; glyceryl stearate; tocopheryl acetate (vitamin E); retinyl palmitate (vitamin A); ascorbyl palmitate (vitamin C); potassium sorbate; phospholipids; hydroxypropyl starch phosphate; benzyl alcohol; sodium pca; palmitic acid; stearic acid; cetearyl glucoside; xanthan gum; methylisothiazolinone; disodium edta; sodium hydroxide; and phenoxyethanol. Such an exfoliating cleanser can also comprise Juice Beauty pure essential oils.

Another example of an organic, juice-based product is a smoothing eye concentrate that can be composed to help reduce puffiness and help brighten the area under the eye. Such an smoothing eye concentrate can comprise a blend of organic grape and carrot juice with essential fatty acids and vitamins B5, C, E, K and P to hydrate, help smooth fine lines, and help brighten the eye area.

Specifically, such a smoothing eye concentrate can comprise an organic juice solution of vitis vinifera (white grape) juice; daucus carota sativa (carrot) juice and aloe barbadensis leaf juice; organic cucumis sativus (cucumber) extract; organic plant oils of helianthus annuus (sunflower) and persea gratissima (avocado); camellia sinensis (organic green tea) leaf extract; organic algae extract; organic essential fatty acids of oenothera biennis (evening primrose); linum usitatissimum (linseed) seed and borago officinali (borage) seed; glycerin; limnanthes alba (meadowfoam) seed oil; phytonadione (vitamin K1); panthenol (vitamin B5); retinyl palmitate (vitamin A); magnesium ascorbyl phosphate (vitamin C); tocopherol (vitamin E); cetearyl glucoside; phenoxyethanol; hesperidin methyl chalcone (vitamin P); sodium hyaluronate; cetearyl alcohol; benzyl alcohol; potassium sorbate; steareth-20; xanthan gum; tetrasodium edta; sodium hydroxide; and citrus aurantium (organge blossom). Such a smoothing eye concentrate can also comprise Juice Beauty pure essential oils.

Another example of an organic, juice-based product is a cleansing gel that can be composed to purify and balance oily, combination, and blemished skin without over drying. Such a cleansing gel can comprise an organic juice solution of citrus medica limonum (lemon) juice, prunus avium (sweet cherry) juice, vitis vinifera (red grape) juice, and aloe barbadensis leaf juice, as well as organic botanical extracts of taraxacum officinale (dandelion) leaf; melissa officinalis (lemon balm) leafand salvia officinalis (sage) leaf; sodium carboxymethyl lauryl glucoside; cocamidopropyl betaine; glycerin; sodium hydroxide; tocopheryl acetate (vitamin E); etinyl palmitate (vitamin A); organic algae extract; potassium sorbate; xanthan gum; sclerotium gum; benzyl alcohol, phenoxyethanol; and disodium edta. Such a cleansing gel can also comprise Juice Beauty pure essential oils.

Another example of an organic, juice-based product is an AHA cleansing gel. Such a cleansing gel can have the following ingredients: Pyrus Malus (Organic Apple) Juice; Citrus Medica Limonum (Organic Lemon) Juice; Vitis Vinifera (Organic Grape) Juice; Aloe Barbadensis (Organic Aloe Vera) Leaf Extract; Water (Aqua); Taraxacum Officinale (Organic Dandelion) Extract; Salvia Officinalis (Organic Sage) Leaf Extract; Citrus Medica Limonum (Organic Lemon) Extract; Organic Honey; Organic Algae Extract; Acetate (Vitamin E Acetate); Retinyl Palmitate (Vitamin A Palmitate); Glycerin, Sodium Carboxymethyl Lauryl Glucoside; Cocamidopropyl Betaine; Sodium Hydroxide; Sclerotium Guy; Tocopheryl Cinnamomum Cassia Leaf (Cinnamon) Oil; Potassium Sorbate; Xanthan Gum; SD Alcohol 40; and Disodium EDTA

Further, the AHA cleansing gel product described above can include juice beauty essential oils, such as May Chang, Ho Wood, and Mandarin. The pure essential oils can be combined in a ratio of 1 to 1 to 5.

Another example of an organic, juice-based product is an exfoliation AHA cleanser. Such a cleanser can have the following ingredients: Pyrus Malus (Organic Apple) Juice; Citrus Medica Limonum (Organic Lemon) Juice; Vitis Vinifera (Organic Grape) Juice; Aloe Barbadensis (Organic Aloe Vera) Leaf Extract; Water (Aqua); Vitis Vinifera (Organic Grape) Seed Extract; Taraxacum Officinale (Organic Dandelion) Extract; Salvia Officinalis (Organic Sage) Leaf Extract; Citrus Medica Limonum (Organic Lemon) Extract; Organic Honey; Organic Algae Extract; Acetate (Vitamin E Acetate); Retinyl Palmitate (Vitamin A Palmitate); Glycerin, Sodium Carboxymethyl Lauryl Glucoside; Cocamidopropyl Betaine; Sodium Hydroxide; Sclerotium Gum; Tocopheryl Cinnamomum Cassia Leaf (Cinnamon) Oil; Potassium Sorbate; Xanthan Gum; SD Alcohol 40; and Disodium EDTA

Further, the cleanser product described above can include juice beauty essential oils, such as May Chang, Ho Wood, and Mandarin. The pure essential oils can be combined in a ratio of 1 to 1 to 5.

Another example of an organic, juice-based product is a peel that can be composed to reveal glowing, healthy skin. The peel product can include the following ingredients: an organic juice solution of pyrus malus (apple) juice, citrus medica limonum (lemon) juice, vitis vinitera (white grape) juice and aloe barbadensis leaf juice, or an organic juice solution of organic alpha/betahydroxy acid juice solution, pyrus malus (apple) juice (malic), citrus medica limonum (lemon) juice (citric), vitis vinifera (white grape) juice (tartaric), organic raw cane sugar (glycolic), organic willow bark extract (salicylic), organic milk peptides (lactic), and organic aloe barbadensis leaf juice.

Such an organic, juice based peel product can also include glycerin; hydroxypropyl starch phosphate; sodium hydroxide; selerotium gum; tocopheryl acetate and tocopherol (vitamin E); ascorbyl palmitate (vitamin C); retinyl palmitate (vitamin A); phospholipids; magnesium aspartate; zinc gluconate; copper gluconate; phenoxyethanol; benzyl alcohol; potassium sorbate; tetrasodium edta; xanthan gum; azulene (natural color); and organic algae extract.

As noted azulene can be added to the ingredients in order to give the serum a green apple appearance.

zinc gluconate is a combination of zinc with a form of glucose (a sugar) that is commonly used in cold lozenges for its antiviral effects. A study reported in Dermatology (2001, volume 203, issue 2, page 40), which is incorporated herein in its entirety as if set forth ion full, evaluated “the place of zinc gluconate in relation to antibiotics in the treatment of acne vulgaris. Zinc was compared to minocycline [an antibiotic ] in a multicenter randomized double-blind trial. 332 patients received either 30 milligrams elemental zinc or 100 milligrams minocycline over 3 months. The primary endpoint was defined as the percentage of the clinical success rate on day 90 . . . . ” The study concluded that “Minocycline and zinc gluconate are both effective in the treatment of inflammatory acne, but minocycline has a superior effect evaluated to be 17% in our study.” Whether or not this relates to topical applications is unknown. Note: High doses of zinc can be toxic. Avoid taking more than 100 mg of zinc per day from a supplement (Source: www.drweil.com). It is also recommended that a daily multivitamin with minerals be taken because increases levels of zinc mean that the body requires more copper and manganese.

copper gluconateis included because copper is an important trace element for human nutrition. The body needs copper to absorb and utilize iron, and copper is also a component of the powerful antioxidant enzyme superoxide dismutase. Copper supplements have been shown to increase superoxide dismutase levels in humans as noted in: Healthnotes Review of Complementary and Integrative Medicine, www.healthnotes.com, which is incorporated herein in its entirety as if set forth in full. The synthesis of collagen and elastin is in part related to the presence of copper in the body, and copper is also important for many other processes. For example, there is research showing that copper is effective for wound healing as noted in: Journal of Clinical Investigation, November 1993, pages 2368-2376: and Federation of European Biochemical Sciences Letter, October 1988, pages 343-346, which are both incorporated herein in their entirety as if set forth in full. However, wound healing is the result of many biophysical processes that have nothing to do with wrinkling.

Phospholipids is a type of lipid (fat) composed of glycerol, fatty acids, and phosphate. Phopholipids are essential to the function of cell membranes by providing a stable surrounding structure. Lecithin and cholesterol are phosopholipids.

Magnesium Aspartate is an amino acid chelate of Magnesium and Aspartic Acid. This complex forms a tight bond and is released in the inner layer of the outer cell membrane.

Amino acids are fundamental constituents of all proteins found in the body, such as: alanine, arginine, asparagines, asparatic acid, cysteine, cystine, glutamic acid, glutamine, glycine, histidine, isoleucine, leucine, lysine, methionine, phenylalanine, proline, serine, threonine, tryptophan, tyrosine, and valine. Some of these amino acids can be synthesized by the body; others, the essential amino acids, must be obtained from protein in the diet. In skin-care products, these types of ingredients work primarily as water-binding agents, and some have antioxidant properties and wound-healing abilities as well. However, these substances cannot affect, change, or rebuild wrinkles. Whether the protein in a skin-care product is derived from an animal or a plant, the skin can't tell the difference.

EDTA is the abbreviation for ethylenediaminetetraacetic acid, which can also be included. It is a stabilizer used in cosmetics to prevent ingredients in a given composition from binding with trace elements (particularly minerals) that can exist in water and other ingredients to cause unwanted product changes such as texture, odor, and consistency problems. The technical term for this function is a chelating agent.

Another example of an organic, juice-based product is a nutrient moisturizer that can be composed to protect and nourish normal and drier skin. Such a nutrient moisturizer can include an organic juice solution of vitis vinifera (white grape) juice, daucus carota sativa (carrot) juice, and aloe barbadensis leaf juice, as well as organic botanical extracts of calendula officinallis flower; matricaria chamomilla flower and tilia europea (linden) leaf; organic rose canina (rosehip) fruit extract; glycerin; organic plant oils, e.g., of butyrospermum parkii (shea butter) and simmondsia chinensis (jojoba) seed; organic essential fatty acids, e.g., of oenothera biennis (evening primrose), linum usitatissimum (linseed) seed, and borago officinali (borage) seed; organic honey; squalane; limnanthes alba (meadowfoam) seed oil; tocopheryl acetate (vitamin E); retinyl palmitate (vitamin A); magnesium ascorbyl phosphate (vitamin C); panthenol (vitamin B5); hyaluronic acid; hydroxypropyl starch phosphate; glyceryl stearate; potassium sorbate; phospholipids; beta carotene; benzyl alcohol; palmitic acid; stearic acid; cetearyl glucoside; xanthan gum; methylisothiazolinone; disodium edta; sodium hydroxide; phenoxyethanol; organic vitis vinifera (grape) seed oil; and organic algae extract. Such a nutrient moisturizer can also comprise Juice Beauty pure essential oils.

Another example of an organic, juice-based product is an oil-free moisturizer that can be composed to protect and nourish normal and drier skin. Such an oil-free moisturizer can include an organic juice solution of pyrus malus (apple) juice, vitis vinifera (white grape) juice, punica granatum (pomegranate) juice, aloe barbadensis seat juice, and cucumis sativus (cucumber) juice, as well as organic botanical extracts of taraxacum officinale (dandelion) leaf; melissa officinalis (lemon balm) leaf and salvia officinalis (sage) leaf, glycerin; alkyl benzoate; retinyl palmitate (vitamin A); panthenol (vitamin B5); tocopheryl acetate (vitamin E); organic beeswax; ascorbyl palmitate (vitamin C); ethylhexyl (octyl) palmitate; caprylic/capric triglyceride; cetearyl alcohol; cetearyl giucoside; sodium pca; sodium hyaluronate; xanthan gum; methylisothiazolinone; disodium edta; sodium hydroxide; phenoxyethanol; and organic algae extract. Such an oil-free moisturizer can also comprise Juice Beauty pure essential oils.

Another example of an organic, juice-based product is redness reducing serum that can be composed to calm and cool redden, inflamed, irritated, and/or highly sensitive skin. Such a redness reducing serum can include an organic juice solution of rubus idaeus (red raspberry) juice, aloe barbadensis leaf juice, and cucumis sativus (cucumber) fruit extract, as well as glycerin; organic botanical extracts of glycyrrhiza glabra (licorice) root; arctostaphylos uva ursi (bearberry) leaf; rubus idaeus (red raspberry) leaf, calendula officinallis flower and matricaria chamomilla flower; organic algae extract; vitis vinifera (organic grape) seed oil; organic essential fatty acids of oenothera biennis (evening primrose); linum usitatissimum (linseed) seed and borago officinali (borage) seed; organic sesamum indicum (sesame) seed oil; tocopherol and tocopheryl (vitamin E); panthenol (vitamin B5); magnesium ascorbyl phosphate (vitamin C); phospholipids; retinyl palmitate (vitamin A); potassium sorbate; magnesium hyaluronic acid; sclerotium gum; phenoxyethanol; benzyl alcohol; disodium edta; sodium hydroxide; and hesperidin methyl chalcone. Such redness reducing serum can also comprise Juice Beauty pure essential oils.

Another example of an organic, juice based skin care product is a sheer moisturizer that can include an active ingredient of Titanium Dioxide (6.25%), as well as organic juice solution of pyrus malus (apple) juice; vitis vinifera (white grape) juice; punica granatum (pomegranate) juice and aloe barbadensis leaf juice; cucumis sativus (cucumber) fruit extract; organic camellia sinensis (green tea) leaf extract; organic cera alba (beeswax); octyl palmitate; caprylic/capric triglyceride; glycerin; cetearyl alcohol; cetearyl glucoside; organic plant oils of helianthus annuus (sunflower); persea gratissima (avocado) and simmondsia chinensis (jojoba) seed, tocopheryl acetate (vitamin E); sodium pca; sodium hyaluronate; retinyl palmitate (vitamin A), phospholipids; panthenol (vitamin B5); ascorbyl palmitate (vitamin C); xanthan gum; tetrasodium edta; phenoxyethanol; methylisothiazolinone; mica and iron oxides (mineral tints); santalum album (sandalwood); and geranium maculatum and citrus tangerina pure essential oils.

Another example of an organic, juice based skin care product is a tinted moisturizer that can include an active ingredient of Titanium Dioxide (6.25%), as well as organic juice solution of pyrus malus (apple) juice; vitis vinifera (white grape) juice; punica granatum (pomegranate) juice and aloe barbadensis leaf juice; cucumis sativus (cucumber) fruit extract; organic camellia sinensis (green tea) leaf extract; organic cera alba (beeswax); octyl palmitate; caprylic/capric triglyceride; glycerin, cetearyl alcohol; cetearyl glucoside; organic plant oils of helianthus annuus (sunflower); persea gratissima (avocado) and simmondsia chinensis (jojoba) seed; tocopheryl acetate (vitamin E); sodium pca; sodium hyaluronate; retinyl palmitate (vitamin A); phospholipids; panthenol (vitamin B5); ascorbyl palmitate (vitamin C); xanthan gum; tetrasodium edta; phenoxyethanol; methylisothiazolinone; santalum album (sandalwood); and geranium maculatum and citrus tangerina pure essential oils.

Another example of an organic, juice-based product is a hydrating mist that can be composed to hydrate and balance skin. Such a hydrating mist can include an organic juice solution of vitis vinifera (white grape) juice , aloe barbadensis leaf juice, and vitis vinifera (red grape) juice, as well as glycerin; organic vitis vinifera (grape) seed oil; tocopheryl acetate and tocopherol (vitamin E); organic rose canina fruit (rose hips) extract; retinyl palmitate (vitamin A); citrus grandis (grapefruit) seed extract; ascorbyl palmitate (vitamin C); phospholipids; lecithin; phenoxyethanol; benzyl alcohol; potassium sorbate; sodium hydroxide; tetrasodium edta; and citric acid.

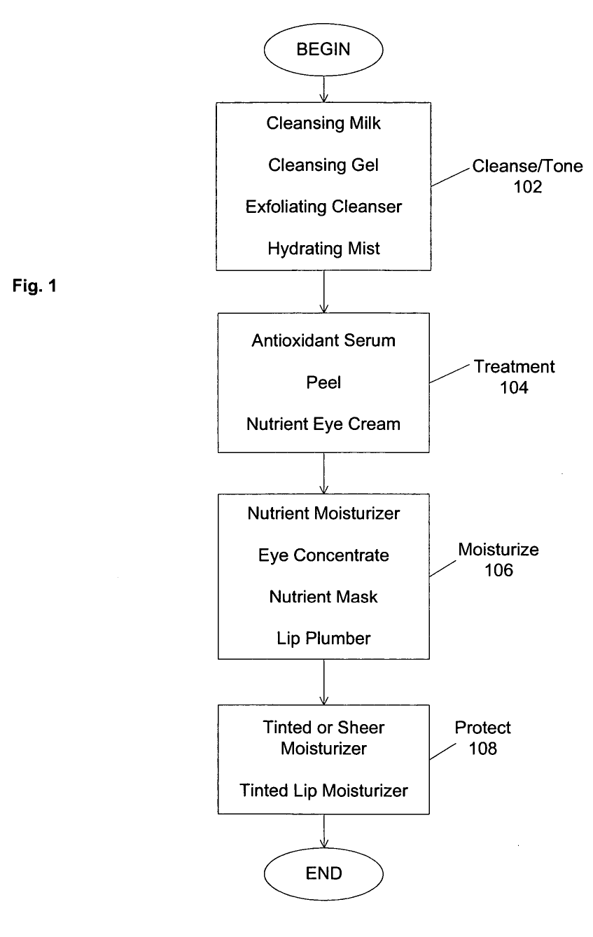

Again, these are just some of the example organic, juice based skin care products that can be composed according the processes described herein. Further, the organic, juice based skin care products can be use din different combinations to achieve different skin treatment solutions as required. FIGS. 1-3 illustrated a few example solutions; however, it will be clear that the number and type of solutions is vast. In each of the solutions depicted in FIGS. 1-3, a cleanse and tone step is followed by a treatment step, a moisturizing step, and a protection step. It will be understood that the cleanse/tone, treatment, and moisturize steps should be performed routinely, e.g., every morning and night, while the protection step can be applied as needed. Further, it will be apparent that steps can be included and or excluded as needed to achieve a certain treatment solution.

In FIG. 1, the cleanse and tone step 102 comprises applying organic, juice based cleansing milk, followed by a cleansing gel, an exfoliating cleanser, and a hydrating mist. Example descriptions of these products can be found above. The treatment step 104 comprises applying one or more organic, juice based antioxidant serums, followed by a peel, and a nutrient eye cream. Again, example descriptions of these products can be found above. The moisturizing step 106 can comprise applying an organic, juice based nutrient moisturizer, followed by a smoothing eye concentrate, a nutrient mask, and lip plumber. Again, example descriptions of these products can be found above. The protective step 108 can comprise applying spf 15 and/or 30 tinted and/or sheer moisturizer, or other moisturizer, and tinted lip moisturizer.

The solution of FIG. 1, can for example, be used to replenish dry, sensitive, and/or aging skin.

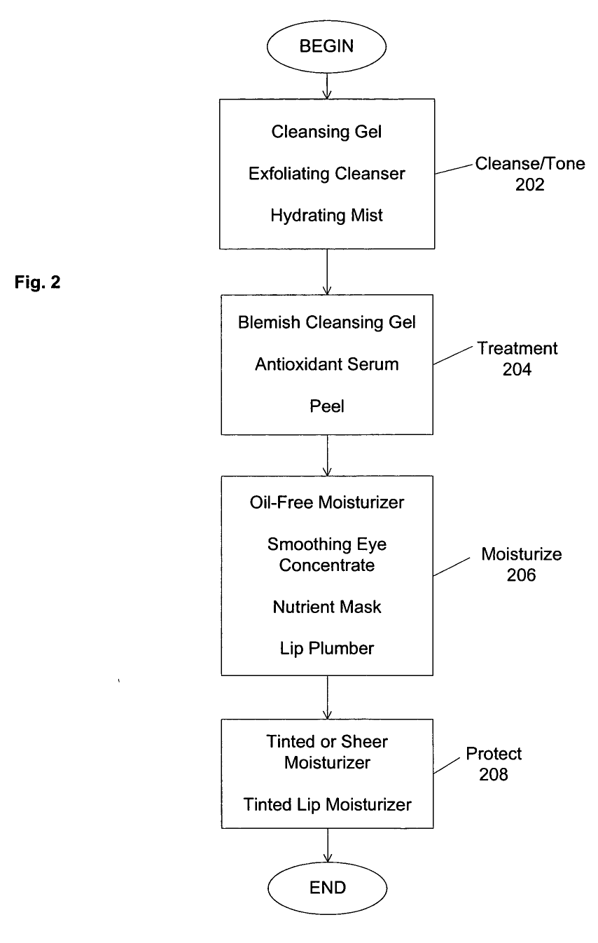

In FIG. 2, the cleanse and tone step 202 comprises applying one or more organic, juice based cleansing gels, followed by an exfoliating cleanser, and a hydrating mist. Example descriptions of these products can be found above. The treatment step 204 comprises applying organic, juice based blemish clearing gel, followed by antioxidant serum, and a peel. Again, example descriptions of these products can be found above. The moisturizing step 206 can comprise applying an organic, juice based oil-free moisturizer, followed by a smoothing eye concentrate, a nutrient mask, and lip plumber. Again, example descriptions of these products can be found above. The protective step 208 can comprise applying spf 15 and/or 30 tinted and/or sheer moisturizer, or other moisturizer, and tinted lip moisturizer.

The solution of FIG. 2, can for example, be used to clarify oily or combination skin problems.

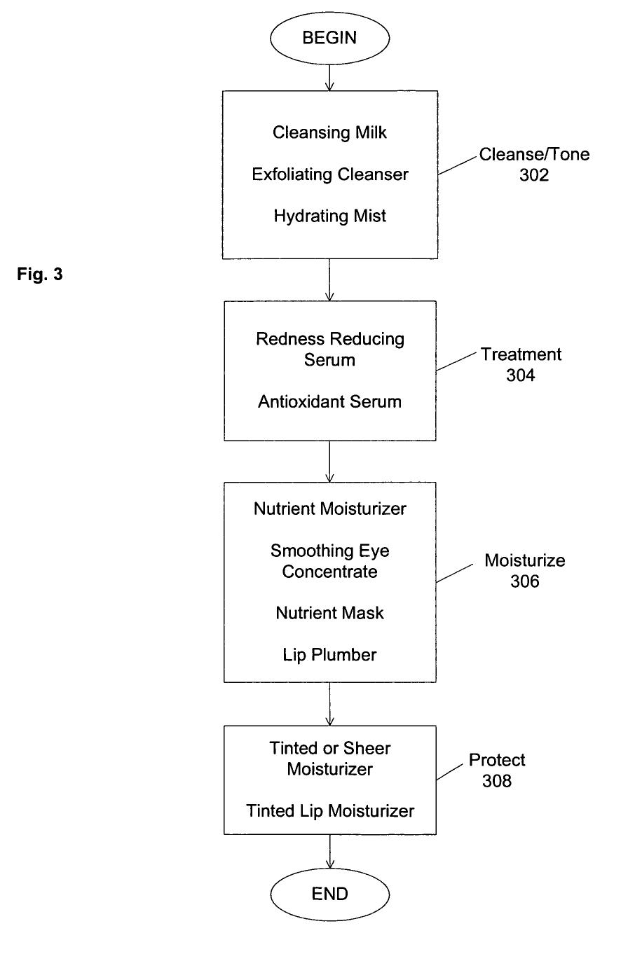

In FIG. 3, the cleanse and tone step 302 comprises applying organic, juice based cleansing milk, followed by an exfoliating cleanser, and a hydrating mist. Example descriptions of these products can be found above. The treatment step 304 comprises applying organic, juice based blemish redness reducing serum followed by an antioxidant serum. Again, example descriptions of these products can be found above. The moisturizing step 306 can comprise applying an organic, juice based nutrient moisturizer, followed by a smoothing eye concentrate, a nutrient mask, and lip plumber. Again, example descriptions of these products can be found above. The protective step 308 can comprise applying spf 15 and/or 30 tinted and/or sheer moisturizer, or other moisturizer, and tinted lip moisturizer.

The solution of FIG. 3, can for example, be used to soothe sensitive, aging, and/or redness prone skin.

While certain embodiments of the inventions have been described above, it will be understood that the embodiments described are by way of example only. Accordingly, the inventions should not be limited based on the described embodiments. Rather, the scope of the inventions described herein should only be limited in light of the claims that follow when taken in conjunction with the above description and accompanying drawings.

What is claimed:

1. An organic, juice based skin care product comprising a blend of organic juices and other organic ingredients, wherein the organic material comprises at least 70% of the juice based skin care product.

2. The organic, juice based skin care product of claim 1, wherein the organic, juice based skin care product does not comprise artificial fillers.

3. The organic, juice based skin care product of claim 1, wherein the organic, juice based skin care product does not comprise toxic preservatives.

4. The organic, juice based skin care product of claim 1, wherein the organic, juice based skin care product does not comprise water to dilute the composition.

5. The organic, juice based skin care product of claim 1, wherein the organic, juice based skin care product does not comprise tars.

6. The organic, juice based skin care product of claim 1, wherein the organic, juice based skin care product does not comprise petroleum.

7. The organic, juice based skin care product of claim 1, wherein the organic, juice based skin care product does not comprise synthetic fragrances.

8. The organic, juice based skin care product of claim 1, wherein the organic, juice based skin care product does not comprise parabens.

9. The organic, juice based skin care product of claim 1, wherein the organic, juice based skin care product is a peel.

10. The organic, juice based skin care product of claim 9, wherein the peel comprises a juice blend of pyrus malus (apple) juice, citrus medica limonum (lemon) juice, vitis vinitera (white grape) juice and aloe barbadensis leaf juice.

11. The organic, juice based skin care product of claim 9, wherein the peel comprises a juice blend of organic alpha/betahydroxy acid juice solution, pyrus malus (apple) juice (malic), citrus medica limonum (lemon) juice (citric), vitis vinifera (white grape) juice (tartaric), organic raw cane sugar (glycolic), organic willow bark extract (salicylic), organic milk peptides (lactic), and organic aloe barbadensis leafjuice.

12. The organic, juice based skin care product of claim 9, wherein the peel comprises glycerin; hydroxypropyl starch phosphate; sodium hydroxide; selerotium gum; tocopheryl acetate and tocopherol (vitamin E); ascorbyl palmitate (vitamin C); retinyl palmitate (vitamin A); phospholipids; magnesium aspartate; zinc gluconate; copper gluconate; phenoxyethanol; benzyl alcohol; potassium sorbate; tetrasodium edta; xanthan gum; azulene (natural color); and organic algae extract.

13. The organic, juice based skin care product of claim 12, wherein the azulene is added in sufficient amounts to provide a green apple appearance fro the peel product.
